(19) United States
(12) Patent Application Publication
(10) Pub. No.: US 2022/0203010 A1
(43) Pub. Date: Jun. 30, 2022

(54) BREASTMILK COLLECTION KIT WITH OPTIMIZED COLOSTRUM COLLECTION

(71) Applicant: MEDELA HOLDING AG, Baar (CH)

(72) Inventors: David Y. Cho, Arlington Heights, IL (US); Shane Einloth, McHenry, IL (US)

(21) Appl. No.: 17/559,501

(22) Filed: Dec. 22, 2021

Related U.S. Application Data
(60) Provisional application No. 63/131,923, filed on Dec. 30, 2020.

(51) Int. Cl. A61M 1/00

(52) U.S. Cl. CPC A61M 1/60

(57) ABSTRACT

A colostrum collection container includes a body having a first opening configured to receive a breastshield and a second opening configured to be operatively coupled to a vacuum generator; a divider sealingly disposed within the body; and an aperture disposed on the divider. The divider is disposed within the body and a first chamber is disposed between the first opening and the divider, and a second chamber is disposed between the divider and the second opening. The aperture fluidly couples the first and second chambers, and a fluid flow path extends from the first opening through the aperture and to the second opening. So configured, a first fluid is collected in the first chamber with gravity assistance while a second fluid flows through the fluid flow path from the first chamber to the second chamber.

### CROSS-REFERENCE TO RELATED APPLICATIONS

This application claims the benefit of U.S. Provisional Application No. 63/131,923 filed Dec. 30, 2021. The entire contents of this application is incorporated herein by reference in its entirety.

### FIELD OF THE DISCLOSURE

The disclosure relates generally to devices and methods used during breastmilk expression. In particular, the disclosure relates to devices and methods used to more efficiently and effectively collect colostrum during breastmilk expression.

### BACKGROUND

Just prior to giving birth, expecting mothers begin producing colostrum vital to the health, growth, and vitality of a newborn. In particular, colostrum typically includes high amounts of bioactive compounds that aid in the health, growth, and vitality of newborn children when compared to mature milk. For example, colostrum contains antibodies that protect the newborn against disease and infection, immune and growth factors, and other bioactives that help to activate a newborn's immune system, jumpstart gut function, and seed a healthy gut microbiome in the first few days of life. However, colostrum is a highly viscous liquid that sticks or otherwise adheres to surface(s) after contact with the surface(s). As a result, colostrum often contacts and adheres to the surfaces of a device used to express breastmilk during breastmilk expression. Disadvantageously, colostrum may be lost during breastmilk expression as the expressed breastmilk flows through the device used to express breastmilk.

### SUMMARY OF THE DISCLOSURE

In accordance with a first aspect, a colostrum collection container includes a body having a first opening configured to receive a breastshield and a second opening configured to be operatively coupled to a vacuum generator; a divider sealingly disposed within the body such that a first chamber is disposed between the first opening and the divider, and a second chamber is disposed between the divider and the second opening; an aperture disposed on the divider such that the aperture fluidly couples the first and second chambers; and a fluid flow path extending from the first opening of the body through the aperture of the divider and to the second opening of the body. In accordance with the first aspect, when a vacuum is created in at least one of the first and second chambers, a first fluid is collected in the first chamber with gravity assistance while a second fluid flows through the fluid flow path from the first chamber to the second chamber.

In accordance with the first aspect, the body may include a first end and a second end opposite the first end. The first opening may be disposed at the first end and the second opening may be disposed at the second end.

In accordance with the first aspect, the first fluid may be more viscous than the second fluid.

In accordance with the first aspect, the first fluid may be one or more of colostrum and/or milk and the second fluid may be at least one of air and/or milk.

In accordance with the first aspect, the body may include a lip that extends radially outward from the first opening.

In accordance with the first aspect, the lip may be integrally formed with the body.

In accordance with the first aspect, the divider may be sealingly coupled to the body proximate the first opening.

In accordance with the first aspect, the divider may include at least one visual indicator that designates a maximum fluid capacity of the body.

In accordance with the first aspect, the aperture may be disposed on the divider such that, when the first fluid in the first chamber is below the at least one visual indicator, the second fluid may flow through the fluid flow path from the first chamber to the second chamber.

In accordance with the first aspect, the aperture may be disposed on the divider such that, when the first fluid in the first chamber is at or above the at least one visual indicator, the first and second fluids may flow through the fluid flow path from the first chamber to the second chamber.

In accordance with the first aspect, a cap may be sealingly coupled to the body. The cap may have a base and a sealing member that extends from the base to at least the aperture when the cap is coupled to the body thereby sealing the aperture.

In accordance with the first aspect, a visual indicator of the at least one visual indicator may be oriented at an angle relative to a vertical axis of the body such that when the body is canted, the visual indicator of the at least one visual indicator designates a maximum fluid capacity of the body.

In accordance with a second aspect, a colostrum collection container includes a body having a first opening configured to receive a breastshield and a second opening; a receptacle sealingly disposed within the body, the receptacle having an aperture, the aperture configured to fluidly couple the first opening and the second opening of the body; and a fluid flow path extending from the first opening through the aperture and to the second opening. In accordance with the second aspect, a first fluid flows through the first opening of the body and is collected in the receptacle with gravity assistance, and a second fluid flows through the first opening, the aperture, and the second opening of the body.

In accordance with the second aspect, the body may include a lip that extends radially outward from the first opening.

In accordance with the second aspect, the lip may be integrally formed with the body.

In accordance with the second aspect, the body may include a first end and a second end opposite the first end. The first opening may be disposed at the first end of the body and the second opening disposed at the second end of the body.

In accordance with the second aspect, the first fluid may be one or more of colostrum and/or milk, and the second fluid may be at least one of air and/or milk.

In accordance with the second aspect, the receptacle may be sealingly disposed in the body proximate the first opening of the body.

In accordance with the second aspect, the receptacle may include at least one visual indicator that designates a maximum fluid capacity of the body.

In accordance with the second aspect, the aperture may be disposed on the receptacle such that when, the first fluid in the receptacle is below the at least one visual indicator, the second fluid may flow from the first opening through the aperture and to the second opening of the body.

In accordance with the second aspect, the aperture may be disposed on the receptacle such that when, the first fluid in the receptacle is at or above the at least one visual indicator, the first and second fluids may flow from the first opening through the aperture and to the second opening of the body.

In accordance with the second aspect, including a cap that sealingly couples to the body. The cap may have a base and a sealing member that extends from the base to at least the aperture when the cap is coupled to the body thereby sealing the aperture.

In accordance with the second aspect, a visual indicator of the at least one visual indicator may be oriented at an angle relative to a vertical axis of the body such that when the body is canted the visual indicator of the at least one visual indicator may designate a maximum fluid capacity of the body.

In accordance with a third aspect, a breastmilk expression system includes a breastshield, a colostrum collection container releasably coupled to the breastshield, and a connector coupled to a second opening of the colostrum collection container. The colostrum collection container includes a body having a first opening that receives the breastshield and the second opening disposed opposite the first opening; a receptacle sealingly disposed within the body, the receptacle having an aperture, the aperture fluidly coupling the first opening and the second opening of the body; and a fluid flow path extending from the first opening through the aperture and to the second opening. In accordance with the third aspect, upon generation of a vacuum in the fluid flow path, a first fluid is collected in the receptacle with gravity assistance and a second fluid flows through the aperture and to the second opening.

In accordance with the third aspect, an overflow container may be releasably received by the connector and a vacuum generator is fluidly coupled to the connector and the colostrum collection container.

In accordance with the third aspect, an assembly guide may be disposed proximate the first opening of the colostrum collection container. The assembly guide may extend radially outward from the first opening and may aid in coupling of the breastshield to the colostrum collection container.

In accordance with the third aspect, the assembly guide may be integrally formed with the body of the colostrum collection container.

In accordance with the third aspect, the body of the colostrum collection container may include a first end and a second end opposite the first end. The first opening may be disposed at the first end and the second opening is disposed at the second end.

In accordance with the third aspect, the first fluid may be one or more of colostrum and/or milk, and the second fluid may be at least one of air and/or milk.

In accordance with the third aspect, the receptacle may be sealingly coupled to the body proximate the first opening.

In accordance with the third aspect, the receptacle may include at least one visual indicator that displays a maximum fill level of the receptacle.

In accordance with the third aspect, the aperture may be disposed on the receptacle such that, when the first fluid disposed in the receptacle is below the at least one visual indicator, the second fluid may flow from the first opening through the aperture and to the second opening of the body.

In accordance with the third aspect, the aperture may be disposed on the receptacle such that, when the first fluid disposed in the receptacle is at or above the at least one visual indicator, the first and second fluids may flow from the first opening through the aperture and to the second opening of the body.

In accordance with the third aspect, a cap may be sealingly coupled to the body. The cap may have a base and a sealing member that extends from the base to at least the aperture when the cap is coupled to the body thereby sealing the aperture.

In accordance with the third aspect, a visual indicator of the at least one visual indicator may be oriented at an angle relative to a vertical axis of the body such that when the body is canted the visual indicator of the at least one visual indicator may designate a maximum fluid capacity of the body.

In accordance with a fourth aspect, a method of collecting colostrum during breastmilk expression includes providing a colostrum collection container including a first opening, a second opening, a receptacle disposed between the first and second openings and having an aperture, and a fluid flow path including the first and second openings and the aperture; and collecting a first fluid in the receptacle of the colostrum collection container with gravity assistance and the vacuum while a second fluid continues to flow from the first opening through the aperture and to the second opening of the colostrum collection container.

In accordance with the fourth aspect, providing the colostrum collection container may further include disposing at least one visual indicator on the body such that a visual indicator of the at least one visual indicator designates a maximum fluid capacity of the body.

In accordance with the fourth aspect, collecting the first fluid may further include collecting the first fluid in the receptacle when the first fluid in the receptacle is below the at least one visual indicator, while the second fluid flows through the fluid flow path from the receptacle to the second opening.

In accordance with the fourth aspect, collecting the first fluid may further include collecting the first fluid in the receptacle until the first fluid in the receptacle is at or above the at least one visual indicator, at which time at least one of the first fluid and the second fluid flows through the fluid flow path from the receptacle to the second opening.

In accordance with the fourth aspect, the method may include coupling a cap to the first opening of the colostrum collection container, the cap having a sealing member that extends to at least the aperture when the cap is coupled to the first opening.

In accordance with the fourth aspect, providing the colostrum collection container may include disposing a visual indicator on the body that is oriented at an angle relative to a vertical axis of the body such that when the body is canted the visual indicator designates a maximum fluid capacity of the body.

In accordance with the fourth aspect, the method may further include creating a vacuum in a portion of the fluid flow path and selectively permitting fluid flow from the first opening to the second opening of the colostrum collection container via the aperture and the vacuum.

In accordance with a fifth aspect, a method of assembling a colostrum collection container including providing a body with a first opening and a second opening; disposing a receptacle within the body and coupling a portion of the receptacle to the body, the receptacle including a wall with an aperture; and forming a fluid flow path including the first and second openings and the aperture of the receptacle, such that when a vacuum is generated in the fluid flow path, a first fluid is permitted to be collected in the receptacle and a second fluid is permitted to flow through the aperture and to the second opening.

In accordance with the fifth aspect, the method may include disposing an assembly guide proximate to the first opening of the body.

In accordance with the fifth aspect, the method may include coupling a breastshield to the first opening of the body using the assembly guide.

In accordance with the fifth aspect, the method may include disposing at least one visual indicator on the body such that a visual indicator of the at least one visual indicator designates a maximum fluid capacity of the body.

In accordance with the fifth aspect, forming the fluid flow path may include forming the aperture on the receptacle such that when the first fluid in the receptacle is below the at least one visual indicator, the second fluid flows from the first opening through the aperture and to the second opening of the body.

In accordance with the fifth aspect, forming the fluid flow path may include forming the aperture on the receptacle such that when the first fluid in the receptacle is at or above the at least one visual indicator, the first fluid and the second fluid flow from the first opening through the aperture and to the second opening of the body.

In accordance with the fifth aspect, the method may include coupling a cap to the first opening of the body, the cap having a sealing member that extends past the aperture when the cap is coupled to the first opening of the body.

### BRIEF DESCRIPTION OF THE DRAWINGS

These and other features and advantages of the various examples disclosed herein will be better understood with respect to the following descriptions and drawings, in which:

FIG. 1 is a block diagram depicting a breastmilk expression system constructed in accordance with the teachings of the present disclosure;

FIG. 2A is a perspective view of an example breastmilk expression system constructed in accordance with the teachings of the present disclosure;

FIG. 2B is a cross-sectional view of the breastmilk expression system of FIG. 2A, taken along the line A-A of FIG. 2A;

FIG. 3 is a perspective, exploded view of the breastmilk expression system of FIG. 2;

FIG. 4 is a perspective view of an example colostrum collection container of FIG. 2;

FIG. 5 is a cross-section of the colostrum collection container of FIG. 4, taken along the line B-B of FIG. 4;

FIG. 6 is a side view of the colostrum collection container of FIG. 4 with a cap coupled thereto;

FIG. 7 is a perspective view of the colostrum collection container and example breastshield of FIG. 2A;

FIG. 8 is a side view of the colostrum collection container and breastshield of FIG. 7;

FIG. 9 is a cross-sectional view of the colostrum collection container, the breastshield, and the connector without an overflow container of FIG. 2, taken along the line A-A of FIG. 2, with fluid flow paths illustrated therethrough when the colostrum collection container is not filled to maximum capacity; and

FIG. 10 is a cross-sectional view of the colostrum collection container, the breastshield, and the connector without an overflow container of FIG. 2, taken along the line A-A of FIG. 2, with fluid flow paths illustrated therethrough when the colostrum collection container is filled to maximum capacity.

### DETAILED DESCRIPTION

A colostrum collection container as disclosed herein collects colostrum during breastmilk expression and maintains a continuous, clinical vacuum while allowing fluid to flow therethrough. The colostrum collection device is coupled to a breastshield and permits fluid flow from the breastshield to a connector. So configured, the colostrum collection device comes into contact with the expressed breastmilk and colostrum shortly after expression, which limits the number of surfaces that the colostrum contacts. Advantageously, because of the colostrum's propensity to stick to surfaces due to the high viscosity of the fluid, the configuration increases the likelihood that the greatest amount of colostrum is collected during breastmilk expression.

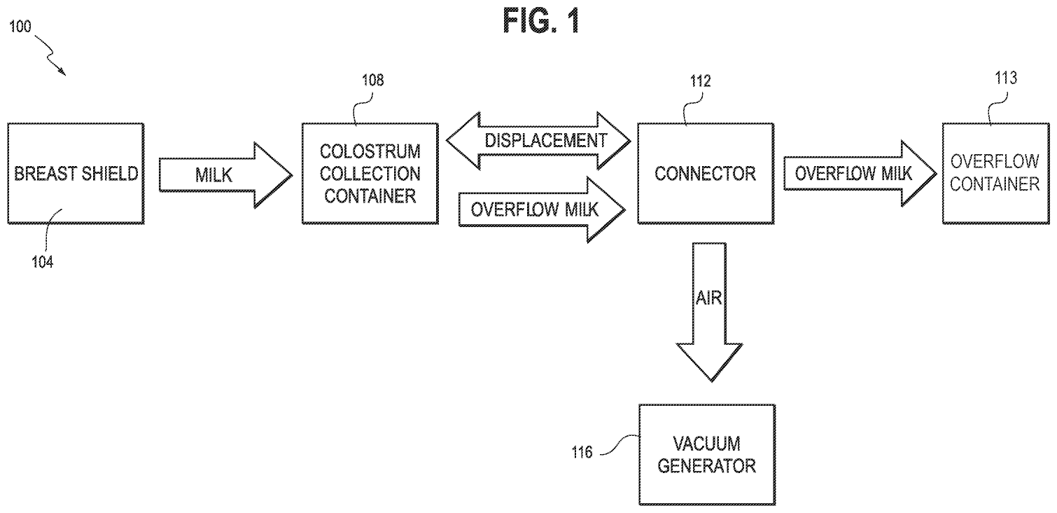

As illustrated in FIG. 1, an example breastmilk expression system 100 as disclosed herein includes a breastshield 104, a colostrum collection container 108, a connector 112, and a vacuum generator 116. The colostrum collection container 108 is releasably coupled to the breastshield 104 and fluidly coupled to the connector 112. The connector 112 is coupled to an overflow container 113, which receives overflow milk during use, for example. The vacuum generator 116 is operably coupled to the connector 112 and the colostrum collection container 108 and is capable of creating a vacuum in at least one of the breastshield 104, the colostrum collection container 108, and the connector 112. To initiate fluid flow through the breastshield 104 and into and through the colostrum collection container 108, the connector 112 transmits the vacuum created by the vacuum generator 116 to the breastshield 104. Accordingly, the vacuum causes a first fluid (e.g., colostrum) to be collected in the colostrum collection container 108 with gravity assistance while a second fluid (e.g., breastmilk, air) to enter the breastshield 104 and pass into the colostrum collection container 108. In one example, the first fluid is one or more of colostrum and/or milk, and the second fluid is at least one of air and/or milk.

The colostrum collection container 108 is configured such that the second fluid passes through the colostrum collection container 108 and into the connector 112, while the first fluid is collected with gravity assistance in the colostrum collection container 108. Such a configuration allows the first fluid to be collected shortly after the expression of the second fluid. Early collection of the first fluid during breastmilk expression advantageously increases the chances that the greatest possible amount of the first fluid is collected because the first fluid is more viscous than the second fluid and has fewer surfaces to interact with before reaching the colostrum collection container 108. Once the colostrum collection container 108 is filled to a maximum capacity with the first fluid, additional quantities of the first fluid expressed with breastmilk flow through the colostrum collection container 108 and into the connector 112. While not illustrated herein, an overflow container may be coupled to the connector 112 that retains the second fluid and additional quantities of the first fluid.

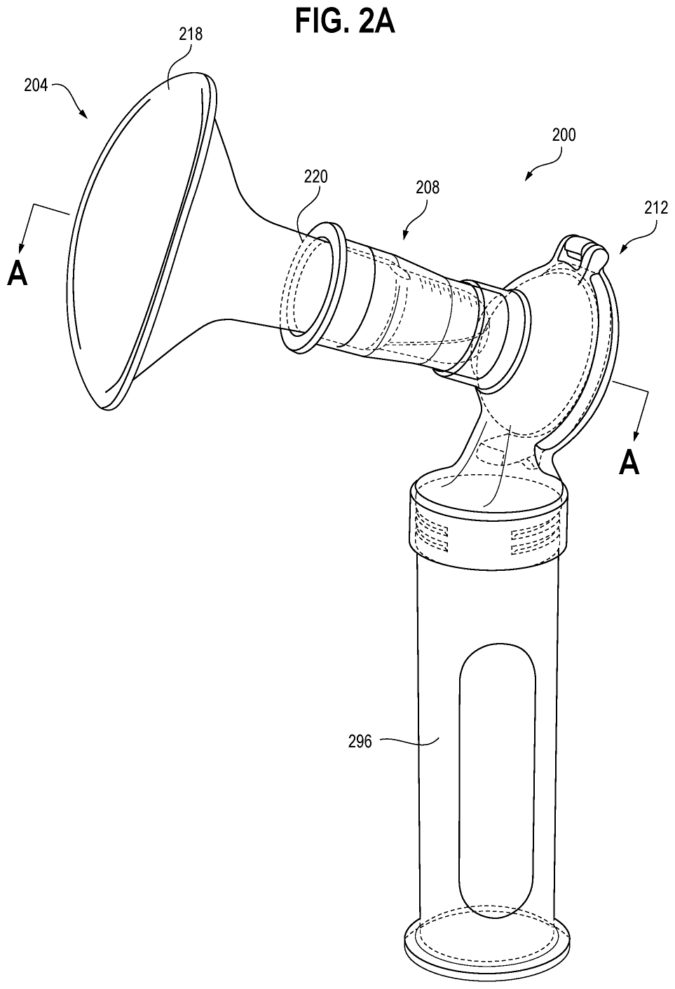

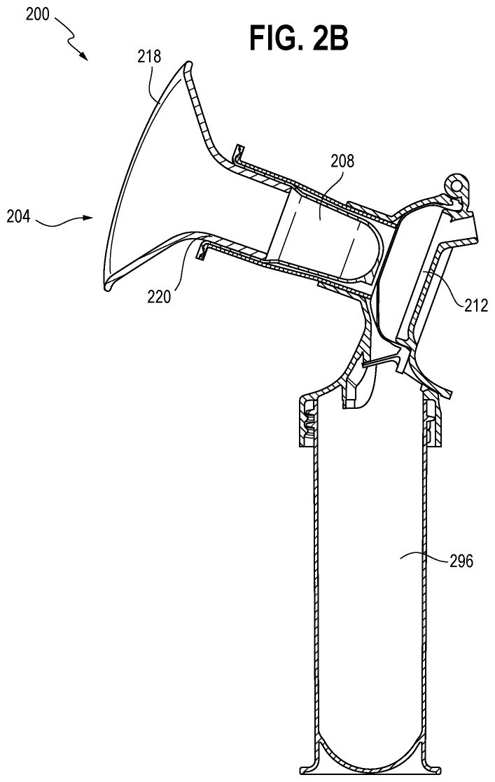

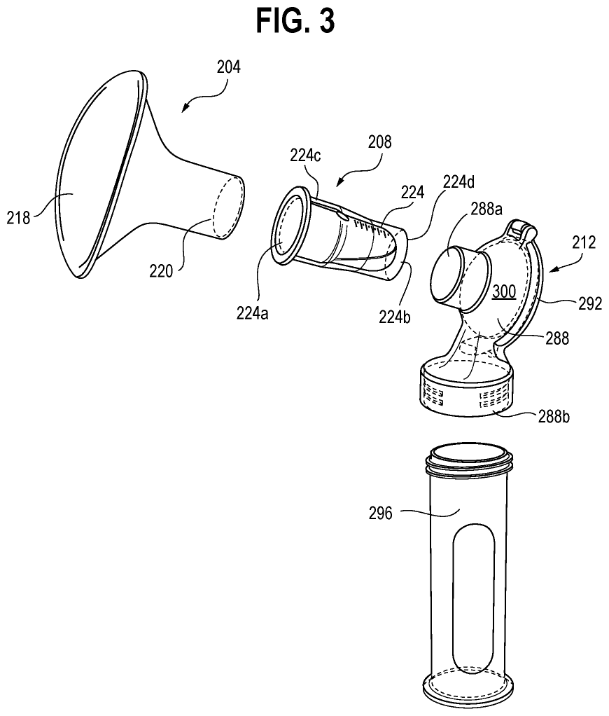

Turning now to FIGS. 2A, 2B, and 3, an example breastmilk expression system 200 is depicted similar to the breastmilk expression system 100 illustrated in FIG. 1, except a vacuum generator is not illustrated, for example. Although not illustrated, it will be understood that a vacuum generator, such as the vacuum generator 116 of FIG. 1, may be operatively coupled to a colostrum collection container of the breastmilk expression system 200 and fall within the scope of the present disclosure. In addition, for ease of reference and to the extent possible, the same or similar components of the breastmilk expression system 200 of FIGS. 2A, 2B, and 3 will retain the same reference numbers as outlined above with respect to the breastmilk expression system 100 of FIG. 1, although reference numbers will be increased by 100.

Much like the breastmilk expression system 100 of FIG. 1, the breastmilk expression system 200 includes a breastshield 204, a colostrum collection container 208, and a connector 212. The colostrum collection container 208 is configured to receive the breastshield 204, and the breastshield 204 is fluidly connected to the colostrum collection container 208 such that fluids may pass from the breastshield 204 to and through the colostrum collection container 208. The breastshield 204 has a generally funnel shaped portion 218 and a sleeve 220 that extends from the funnel portion 218. The funnel portion 218 is shaped and sized for being received onto a breast. The sleeve 220, e.g., a nipple tunnel, conducts the second fluid into the colostrum collection container 208 and extends into the colostrum collection container 208 for a distance. So configured, the colostrum collection container 208 may be a second surface, if not a first surface, on which the first fluid and the second fluid contact during expression. This minimizes, if not mitigates, the viscous first fluid from sticking to other surfaces before being collected. The connector 212 is also operatively connected to an overflow container 296.

As depicted in FIG. 3, the colostrum collection container 208 includes a body 224 having a first opening 224a, which is configured to receive the breastshield 204, and a second opening 224b, which is configured to be received by and coupled to the connector 212. The second opening 224b is also configured to be operably coupled to a vacuum generator (e.g., the vacuum generator 116 of FIG. 1), such as by way of the connector 212. The body 224 also includes a first end 224c and a second end 224d opposite the first end 224c. The first opening 224a is disposed at the first end 224c and the second opening 224b is disposed at the second end 224d.

In addition, the connector 212 includes a housing 288, a first opening 288a, a second opening 288b, and a valve mechanism 292 disposed, at least in part, within the housing 288 between the first and second openings 288a, 288b. The valve mechanism 292 can be, for example, any valve mechanism as is known in the art. The second end 288b of the connector 212 may include threads or any suitable mechanism for releasable attachment to the overflow container 296, such as a milk bottle or the like. The connector 212 also includes a channel 300 for conducting expressed breastmilk from the colostrum collection container 208 and into the container 296.

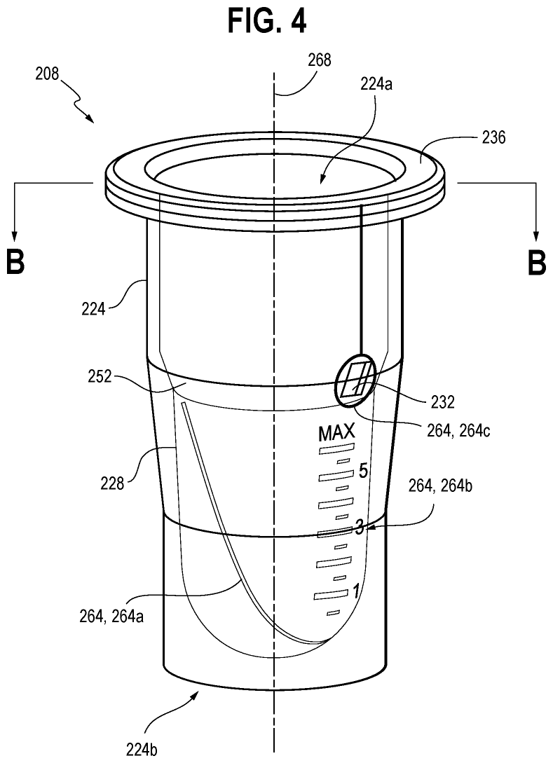

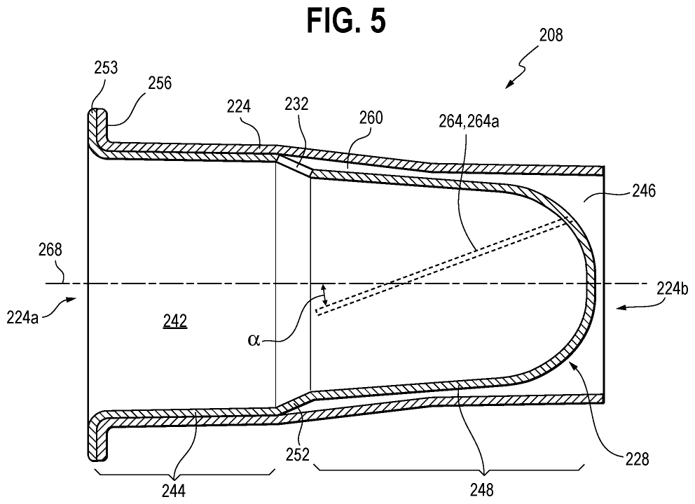

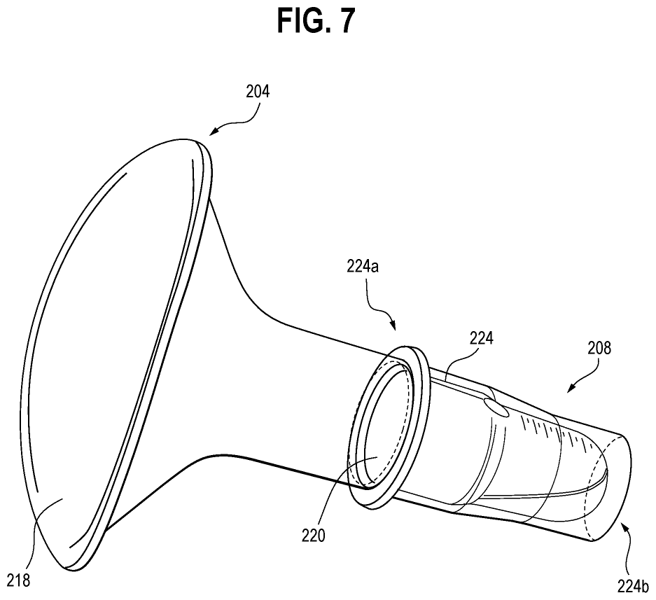

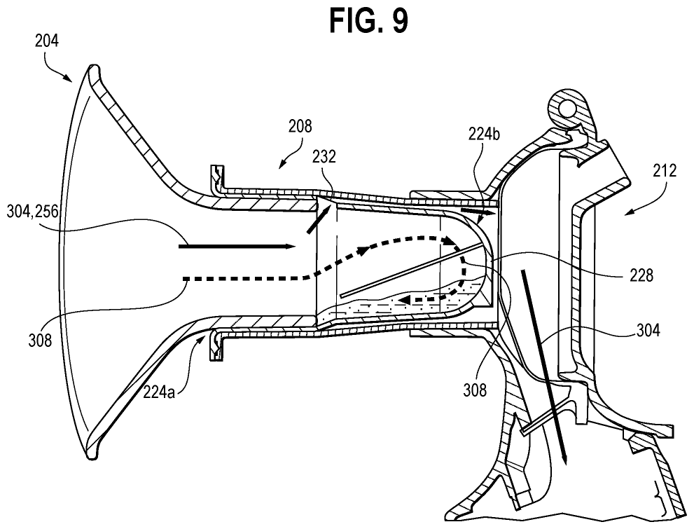

As best illustrated in FIGS. 4 and 5, the colostrum collection container 208 includes a divider 228, such as a receptacle, sealingly disposed within the body 224 proximate the first opening 224a of the body 224, and an aperture 232 disposed on the divider 228. As illustrated in FIG. 7, the first opening 224a of the body 224 is sized to receive an outer diameter of the sleeve 220 of the breastshield 204. The second opening 224b is sized to be received by the connector 212 (see, e.g., FIG. 9) and can, for example, be of a smaller diameter than the first opening 224a. The body 224 further includes an assembly guide 234 that is disposed proximate the first opening 224a of the colostrum collection container and may guide the breastshield 204 into and through the first opening 224a. The assembly guide 234 may extend radially outward from the first opening 224a and may be configured to aid coupling of the breastshield 204 to the colostrum collection container 208. Additionally, the assembly guide 234 may be integrally formed with the body 224 of the colostrum collection container 208. For example, as illustrated in FIGS. 4 and 5, the assembly guide 234 can be a lip 236 that extends radially outward from the first opening 224a. In such an example, the lip 236 may be integrally formed with the body 224. Advantageously, the lip 236 may prevent a user from coupling the first opening 224a of the body 224 to the connector 212, and potentially make for an easier assembly of the breastmilk expression system 200.

The divider 228 is sealingly coupled to the body 224 proximate the first opening 224a and extends from the first opening 224a toward the second opening 224b of the body 224. As illustrated in FIG. 5, the example divider 228 has a first section 244, a second section 248, an angled surface 252, and a lip 253. The first section 244 is disposed proximate the first opening 224a of the body 224 and is substantially parallel to the body 224. For example, the first section 244 can be integrally formed with the lip 253, as depicted in FIG. 5. The angled surface 252 extends radially inward from the first section 244 toward the second section 252. The second section 248 extends from the angled surface 252 toward the second end 224b of the body 224 at an angle.

Importantly, and as depicted in FIG. 5, the divider 228 is sealingly disposed within the body 224 such that the divider 228 creates a first chamber 242 and a second chamber 246. In particular, the divider 228 is sealingly disposed within the body 224 such that the first chamber 242 is disposed between the first opening 224a of the body 224 and the divider 228. The second chamber 246 is disposed between the divider 240 and the second opening 224b of the body 224. As will be discussed further, when a vacuum is created (e.g., such as by the vacuum generator 116 of FIG. 1) in at least one of the breastshield 204, the colostrum collection container 208, or the connector 212, a first fluid (e.g., colostrum, breastmilk) is collected in the first chamber 242 with gravity assistance while a second fluid (e.g., breastmilk and/or air, etc.) flows through a fluid flow path from the first chamber 242 to the second chamber 246. Advantageously, the first chamber 242 is configured to initially first come into contact with the expressed breastmilk and the colostrum. In doing so, the first chamber 242 may be the first surface on which the colostrum contacts, which increases the likelihood that the most amount of colostrum is collected. Moreover, in one example, the first fluid is one or more of colostrum and/or milk, and the second fluid is at least one of air and/or milk.

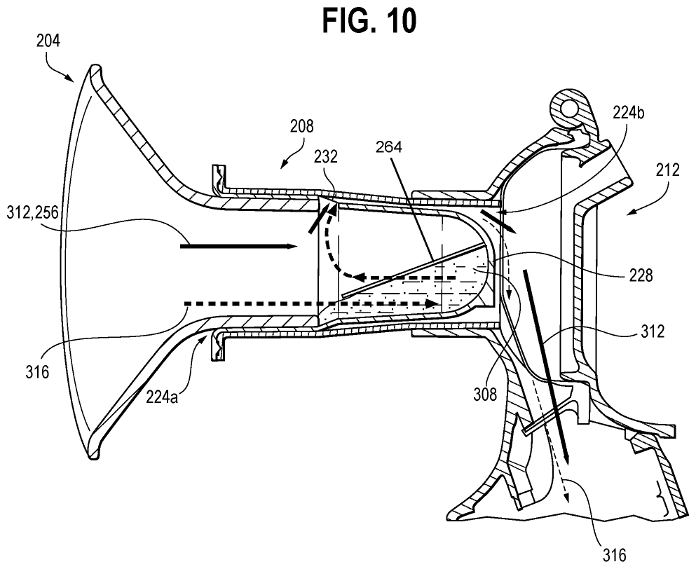

The aperture 232 is disposed on the divider 228 such that a fluid flow path 256 (FIGS. 9 and 10) is formed from the first opening 224a through the aperture 232 and to the second opening 224b. In particular, the aperture 232 creates a fluid flow path 256 from the first chamber 242 to the second chamber 246. The aperture 232 may be disposed anywhere on the divider 232 so that the fluid flow path 256 is formed from the first opening 224a to the second opening 224b. For example, as illustrated in FIGS. 4 and 5, the aperture 232 can be disposed on the angled surface 252 between the first and second sections 244, 248. In such an example, a gap 260 is formed between the divider 228 and the body 224 of the colostrum collection container 108 and provides fluid communication between the aperture 232 and the second opening 224b of the body 224. As such, fluid flowing into the first opening 224a of the body 224 and through the aperture 232 can flow to and through the second opening 224b of the body 224. Further, the aperture 232 may be disposed on the divider 228 such that the aperture 232 is parallel with the divider 228. For example, the aperture 232 can be disposed on the angled surface 252 of the divider 228 such that the aperture 232 is parallel to the angled surface 252.

The aperture 232 and the gap 260 cooperate to selectively permit fluid flow from the first opening 224a of the body 224 of the colostrum collection container 208 to the second opening 224b of the body 224. In particular, the orientation, size, and placement of the aperture 232 and the size and the placement of the gap 260 can be configured such that fluid flows in only one direction. In other words, in this example, fluid flows from the first opening 224a of the body 224 of the colostrum collection container 208 through the aperture 232 and to the second opening 224b of the body 224, but fluid cannot flow from the second opening 224b through the aperture 232 and into the colostrum collection container 208.

As depicted in FIG. 4, at least one visual indicator 264 is disposed on the colostrum collection container 208 and indicates various fluid levels in the colostrum collection container 208 depending on the orientation of the colostrum collection container 208. For example, a first visual indicator 264a of the at least one visual indicator 264 can be disposed on the divider 228 of the colostrum collection container 208. The first visual indicator 264a is oriented at an angle α relative to a vertical axis 268 of the colostrum collection container 208 that is less than 90 degrees such that when the breastmilk expression system 200 is in use, the first visual indicator 264a designates a maximum fluid capacity of the colostrum collection container 208. In other words, when the colostrum collection container 208, and more particularly the body 224 of the colostrum collection container 208, is canted the first visual indicator 264a of the at least one visual indicator 264 designates a maximum fluid capacity of body 224.

As further depicted in FIG. 4, the at least one visual indicator 264 may also include a second visual indicator 264b and a third visual indicator 264c. The second visual indicator 264b may be disposed on the body 224 or the divider 228 of the colostrum collection container 208 and designates various fluid levels within the colostrum collection container 208. For example, as illustrated in FIG. 4, the second visual indicator 264b is disposed on the divider 228 of the colostrum collection container 208 and includes various markings that extend along a height of the divider 228. The second visual indicator 264b is similar to the first visual indicator 264a in that the second visual indicator 264b also designates a maximum fluid capacity of the colostrum collection container 208. Specifically, the second visual indicator 264b includes a plurality of markings associated with an amount of fluid within the divider 228, such as 1 ounce, 3 ounces, 5 ounces, and a maximum amount MAX, in this example. However, the second visual indicator 264b is different from the first visual indicator 264a in that the second visual indicator is substantially parallel to the vertical axis 268 of the colostrum collection container 208. Further, the second visual indicator 264b designates a maximum fluid capacity of the colostrum collection container 208 when the colostrum collection container is oriented in a substantially vertical manner. In other words, the second visual indicator 264b designates a maximum fluid capacity of the colostrum collection container 208 when the colostrum collection container 208 is upright and, for example, stored.

The third visual indicator 264c may also be disposed on either the body 224 or the divider 228 of the colostrum collection container 208 and designates a location of the aperture 232. For example, as illustrated in FIG. 4, the third visual indicator 264c is disposed on the body 224 of the colostrum collection container 208. The third visual indicator 264c may be any size and shape that draws the attention of a user and makes the location of the aperture 232 easily identifiable. For example, the third visual indicator 264c can be a circle disposed on the body 224 of the colostrum collection container 208 and substantially surrounds the aperture 232. So configured, a user can use the third visual indicator 264c to locate the aperture 232 in the event that fluid flow from the first opening 224a to the second opening 224b of the body 224 is interrupted. As such, the user can then determine if a blockage in the aperture 232 is preventing fluid flow from the first opening 224a to the second opening 224b of the body 224.

The at least one visual indicator 264a, 264b, 264c may designate the location of the aperture 232. This may aid a user in locating the aperture 232 in the event that there is an interruption in fluid flow through the colostrum collection device 208. For example, the aperture 232 can be disposed on the divider 228 such that when the first fluid disposed in the first chamber 242 is below the at least one visual indicator 264a, 264b, 264c, the second fluid flows through the fluid flow path from the first chamber 242 to the second chamber 246. In other examples, the aperture 232 can be disposed on the divider 228 such that when the first fluid disposed in the first chamber 242 is at or above the at least one visual indicator 264a, 264b, 264c, the first and second fluids flow through the fluid flow path from the first chamber 242 to the second chamber 246.

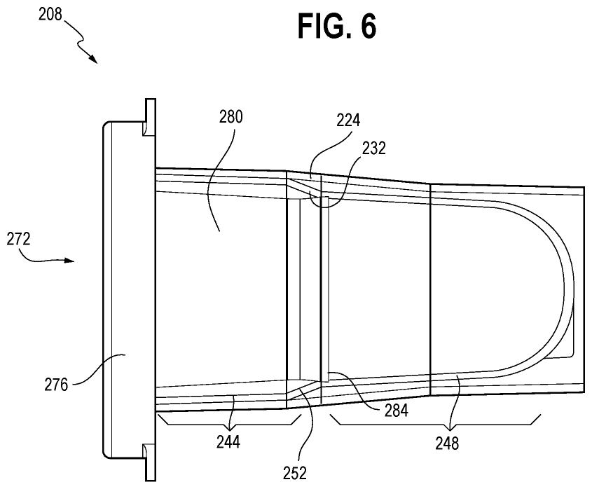

Referring now to FIG. 6, a cap 272 may be configured to be sealingly coupled to the body 224 of the colostrum collection container 208. For example, the cap 272 can be sealingly coupled to the body 224 over the first opening 224a of the body 224 via a snap fit. However, in other examples, the cap 272 can be sealingly coupled to the colostrum collection container 208 via a threaded connection. In any event, the cap 272 includes a base 276 that couples to the body 224 and a sealing member 280 that extends from the base 276. In particular, the sealing member 280 extends from the base 276 to at least the aperture 232 when the cap 272 is coupled to the body 224 thereby sealing the aperture 232. So configured, fluid cannot flow through the aperture 232 when the cap 272 is coupled to the body 224, which allows the colostrum collection container 208 to be transported throughout a medical facility and stored within the medical facility without the concern that colostrum retained within the colostrum collection container 208 will be spilled or otherwise lost. In particular, the sealing member 280 includes a sealing surface 284 that engages the divider 240 thereby creating a seal preventing colostrum stored within the colostrum collection container 208 from passing through the aperture 232.

While the colostrum collection container 208 has been discussed as being slidably received by the connector 212, the colostrum collection container 208 may be coupled to the connector 212 in a different manner. For example, the second opening 224b of the colostrum collection container 208 can be threaded and the first opening 288a of the connector 212 can be threaded in a manner complimentary to the threads of the second opening 224b. In such an example, the threaded nature of the colostrum collection container 208 and the connector 212 can reduce the likelihood that the colostrum collection container 208 and the connector 212 disconnect during use. Advantageously, such a configuration could reduce, if not eliminate, spilling the colostrum retained in the colostrum collection container 208.

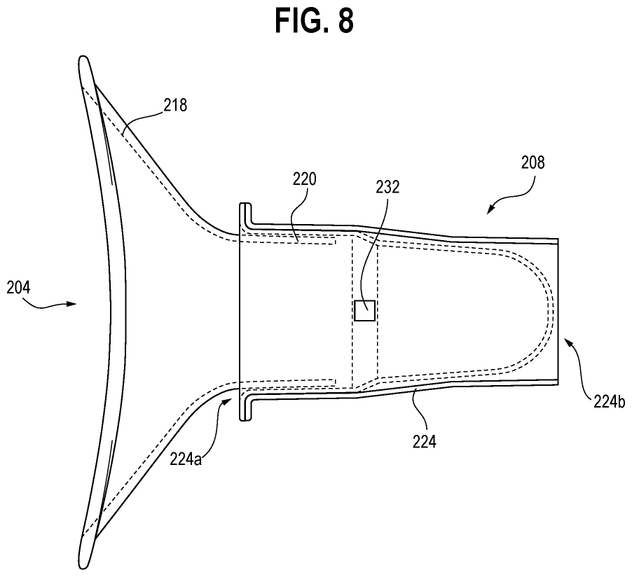

FIGS. 7 and 8 illustrate another view of the breastshield 204 releasably received by the colostrum collection container 208. In particular, the first opening 224a of the colostrum collection container 208 slidably receives the sleeve 220, e.g., a nipple tunnel, of the breastshield 204. The sleeve 220 extends into the body 224 of the colostrum collection container 208 for a distance. So configured, the sleeve 220 conducts the first and second fluids into the colostrum collection container 208. As best seen in FIG. 8, the aperture 232 is disposed on the divider 228. In particular, and in this example, the aperture 232 is disposed on the angled surface 252 of the divider 228 between the first and second sections 244, 248. So configured, the first and/or second fluids can flow from the sleeve 220 of the breastshield 204 into the colostrum collection container 204, through the aperture 232 and out of the second opening 224b of the colostrum collection container 204.

Turning now to FIGS. 9 and 10, an example of the fluid flow path 256 through the breastmilk expression system 200 is depicted. Generally, a vacuum created by the vacuum generator (e.g., the vacuum generator 116 of FIG. 1) causes a flow of a fluid through the colostrum collection container 208. In addition, the size and position of the aperture 232 and the amount of the first fluid (e.g., colostrum) in the colostrum collection container 208 determines which fluid, or fluids, will flow through the colostrum collection container 208, for example, as explained more below. In addition, in one example, the first fluid is one or more of colostrum and/or milk, and the second fluid is at least one of air and/or milk.

Referring now to FIG. 9, a first set of arrows 304 and a second set of arrows 308 generally illustrate fluid flow through the breastmilk expression system 200, when the colostrum collection container 208 is not filled to maximum capacity. In particular, the first set of arrows 304, which are in solid line form, depict a vacuum flow path and the fluid flow path of the second fluid (e.g., breastmilk and air). In addition, the second set of arrows 308, which are in dashed line form, depict the fluid flow path of the first fluid (e.g., colostrum and/or milk). In one example, the fluid flow path of each of the first and second fluids is initially the same, such as when the first and second fluids flow both flow through the first opening 224a of the body 224, as explained more below. Once the vacuum is created in the breastshield 204 thereby expressing breastmilk, the second fluid (e.g., expressed breastmilk and/or air) flows from the breastshield 204 to and through the colostrum collection container 208 and into the connector 212, as depicted by the first set of arrows 304. As the second fluid (e.g., the expressed breastmilk and/or the air) flows through the breastmilk expression system 200 (the first set of arrows 304), the first fluid (e.g., the colostrum and/or breastmilk) expressed is retained within the colostrum collection container 208 with gravity assistance, as depicted by the second set of arrows 308 until the first fluid (e.g., the colostrum) reaches a maximum capacity, as explained more below. Said another way, and in one example, upon generation of the vacuum in the fluid flow path 256, the first fluid flows through the first opening 224a of the body 224 and is collected in the receptacle 228 with gravity assistance, and the second fluid flows through the first opening 224a, the aperture 232 of the receptacle 228, and the second opening 224b of the body. This occurs when the first fluid (e.g., the colostrum) disposed in the receptacle 228 is below the at least one visual indicator 264, which in this example is the visual indicator 264a (see also FIG. 5). Once the first fluid is at or exceeds the at least one visual indicator 264, for example, the first fluid has a different flow path, as explained more below.

More specifically and referring now to FIG. 10, a third set of arrows 312 and a fourth set of arrows 316 that illustrate fluid flow through the breastmilk expression system 200, when the colostrum collection container 208 is filled to a maximum capacity, are depicted. In particular, the third set of arrows 312, 256, which are in solid line form, depicts a vacuum flow path and the fluid flow path of the second fluid (e.g., the breastmilk, the air). In addition, the fourth set of arrows 316, which are in dashed line form, depicts the flow of the first fluid (e.g., colostrum). As depicted in FIG. 10, the fluid flow through the breastmilk expression system 200 when the amount of the first fluid is equal to or greater than the maximum capacity is different from the fluid flow in FIG. 9 when the amount of the first fluid (e.g., colostrum) in the colostrum collection container 108 is less than the maximum fluid capacity, such as less than the at least one visual indicator 264.

In particular, once the vacuum is created in the breastshield 204 thereby expressing breastmilk, the second fluid (e.g., the expressed breastmilk) flows from the breastshield 204 to and through the colostrum collection container 208, and into the connector 212, as depicted by the third set of arrows 312. As the second fluid (e.g., the expressed breastmilk, the air) flows through the breastmilk expression system 200 (the third set of arrows 312), the first fluid (e.g., the colostrum) initially expressed along with the second fluid (e.g., the breastmilk) flows from the breastshield 204, into the colostrum collection container 208, through the aperture 232, and into the connector 212, as depicted by the fourth set of arrows 316.

More specifically, and in this example, when the first fluid (e.g., colostrum) disposed in the receptacle 228 is at or above the at least one visual indicator 264, the first and second fluids flow from the first opening 224a through the aperture 232 and to the second opening 224b of the body 224. In one example, the first fluid may initially flow from the first opening 224a of the body 224 and into the receptacle 228, but then ultimately flows through the aperture 232 and to the second opening 224b, which leads to the connector 212, as explained above.

In view of the foregoing, it is understood that the colostrum collection container 108, 208 used in the breastmilk expression system 100, 200 may employ the following method for collecting colostrum during breastmilk expression. For example, the method can include providing the colostrum collection container 108, 208 that includes the first opening 224a, the second opening 224b, a receptacle 228 disposed between the first and second openings 224a, 224b and having the aperture 232, and the fluid flow path 256 including the first opening 224a, the second opening 224b, and the aperture 232. Once the colostrum collection container 108, 208 is provided, the method includes creating a vacuum in a portion of the fluid flow path 256 and selectively permitting fluid flow from the first opening 224a to the second opening 224b of the colostrum collection container 108, 208 via the aperture 232 and the vacuum. When the vacuum is created, the method then includes collecting the first fluid in the receptacle 228 of the colostrum collection container 108, 208 with gravity assistance and the vacuum while the second fluid continues to flow from the first opening through the aperture 232 and to the second opening 224b of the colostrum collection container 108, 208.

In the example method of collecting colostrum discussed above, providing the colostrum collection container 108, 208 can further include disposing the at least one visual indicator 26a, 264b, 264c on the body 224 such that a visual indicator of the at least one visual indicator 264a, 264b, 264c designates a maximum fluid capacity of the body 224. The example method of collecting colostrum can further include collecting the first fluid in the receptacle 228 when the first fluid in the receptacle 228 is below the at least one visual indicator 264a, 264b, 264c while the second fluid flows through the fluid flow path 256 from the receptacle 228 to the second opening 224b. The example method of collecting colostrum can also include collecting the first fluid in the receptacle 228 until the first fluid in the receptacle 228 is at or above the at least one visual indicator 264a, 264b, 264c, at which time at least one of the first fluid and the second fluid flows through the fluid flow path 256 from the receptacle 228 to the second opening 224b.

The example method of collecting colostrum can further include coupling a cap 272 to the body 224 of the colostrum collection container 104, 204. The cap 272 has the base 276 and the sealing member 280 that extends from the base 276 to at least the aperture 232 when the cap 272 is coupled to the body 224 thereby sealing the aperture 232. In some examples, providing the colostrum collection container 108, 208 can further include disposing the visual indicator 264a, 264b, 264c on the body 224 that is oriented at an angle α relative to a vertical axis 268 of the body 224 such that when the body 224 is canted, the visual indicator 264a, 264b, 264c designates a maximum fluid capacity of the body 224.

In view of the foregoing, it is understood that the colostrum collection container 108, 208 used in the breastmilk expression system 100, 200 may employ the following method for assembling the colostrum collection container 108, 208. For example, the method of assembling the colostrum collection container 108, 208 can include providing the body 224 with the first opening 224a and the second opening 224b; disposing the receptacle 228 within the body 224 and coupling a portion of the receptacle 228 to the body 224, the receptacle 228 including a wall 228a with an aperture 232; and forming the fluid flow path 256 including the first and second openings 224a, 224b and the aperture 232 of the receptacle 228, such that when a vacuum is generated in the fluid flow path 256, the first fluid is permitted to be collected in the receptacle 228 and the second fluid is permitted to flow through the aperture 232 and to the second opening 224b.

The example method of assembling the colostrum collection container 108, 208 discussed above can further include disposing the assembly guide 234 proximate to the first opening 224a of the body 224. In some examples, the method can further comprise coupling a breastshield 104, 204 to the first opening 224a of the body 224 using the assembly guide 234. The example method of assembling the colostrum collection container 108, 208 can further include disposing the at least one visual indicator 264a, 264b, 264c on the body 224 such that a visual indicator of the at least one visual indicator 264a, 264b, 264c designates a maximum fluid capacity of the body 224.

Moreover, forming the fluid flow path 256 described in the example method of assembling the colostrum collection container 108, 208 can further include forming the aperture 232 on the on the receptacle 228 such that when the first fluid in the receptacle 228 is below the at least one visual indicator 264a, 264b, 264c, the second fluid flows from the first opening 224a through the aperture 232 and to the second opening 224b of the fluid flow path 256. In some examples, forming the fluid flow path 256 includes forming the aperture 232 on the receptacle 228 such that when the first fluid in the receptacle 228 is at or above the at least one visual indicator 264a, 264b, 264c, the first and the second fluid flow from the first opening 224a through the aperture 232 and to the second opening 224b of the fluid flow path 256. The method of assembly, in some examples, can further include coupling a cap 272 to the body 224. The cap 272 having a base 276 and a sealing member 280 that extends from the base 276 to at least the aperture 232 when the cap 276 is coupled to the body 224.

In view of the foregoing, it will be understood that the breastmilk expression system 100, 200 and related methods of the present disclosure have several advantages. For example, the aperture 232 of the colostrum collection container 108, 208 forms a fluid flow path 256 from the first opening 224a through the aperture 232 and to the second opening 224b, which can maintain a clinical vacuum and selectively permit fluid flow of a second fluid (e.g., breastmilk, air) through the aperture 232 while retaining the first fluid (e.g., colostrum) in the colostrum collection container 108, 208. As another example, placement of the colostrum collection container 108, 208 within the breastmilk expression system 100, 200 allows the colostrum expressed during breastmilk expression to contact the least amount of surfaces throughout the breastmilk expression system 100, 200, which can increase the amount of colostrum collected. As yet another example, the assembly guide 234 of the colostrum collection container 108, 208 aids in assembly of the breastmilk expression system 100, 200 by selectively permitting the colostrum collection container 108, 208 to receive certain other components of the breastmilk expression system 100, 200. Finally, as another example, the cap 272 that is sealingly coupled to the body 224 of the colostrum collection container 108, 208 can seal the aperture 232 and securely retain the collected colostrum in the colostrum collection container 108, 208 during transportation and storage, providing yet another benefit.

While various examples have been described above, this disclosure is not intended to be limited thereto. Variations can be made to the disclosed examples that are still within the scope of the appended claims.

1. A colostrum collection container comprising:
a body having a first opening configured to receive a breastshield and a second opening configured to be operably coupled to a vacuum generator;
a divider sealingly disposed within the body such that a first chamber is disposed between the first opening and the divider, and a second chamber is disposed between the divider and the second opening;
an aperture disposed on the divider such that the aperture fluidly couples the first and second chambers; and
a fluid flow path extending from the first opening of the body through the aperture of the divider and to the second opening of the body,
where, when a vacuum is generated in at least one of the first and second chambers, a first fluid is collected in the first chamber with gravity assistance while a second fluid flows through the fluid flow path from the first chamber to the second chamber.

2. The colostrum collection container of claim 1, wherein the body includes a first end and a second end opposite the first end, the first opening disposed at the first end and the second opening disposed at the second end.

3. The colostrum collection container of claim 1, wherein the first fluid is more viscous than the second fluid.

4. The colostrum collection container of claim 1, wherein the first fluid comprises one or more of colostrum and/or milk, and the second fluid comprises at least one of air and/or milk.

5. The colostrum collection container of claim 1, wherein the body includes a lip that extends radially outward from the first opening.

6. The colostrum collection container of claim 5, wherein the lip is integrally formed with the body.

7. The colostrum collection container of claim 1, wherein the divider is sealingly coupled to the body proximate the first opening.

8. The colostrum collection container of claim 1, wherein the divider includes at least one visual indicator that designates a maximum fluid capacity of the body.

9. The colostrum collection container of claim 8, wherein the aperture is disposed on the divider such that, when the first fluid in the first chamber is below the at least one visual indicator, the second fluid flows through the fluid flow path from the first chamber to the second chamber.

10. The colostrum collection container of claim 8, wherein the aperture is disposed on the divider such that when the first fluid disposed in the first chamber is at or above the at least one visual indicator, the first and second fluids flow through the fluid flow path from the first chamber to the second chamber.

11. The colostrum collection container of claim 1, further comprising a cap that is configured to be sealingly coupled to the body, the cap having a base and a sealing member that extends from the base to at least the aperture when the cap is coupled to the body thereby sealing the aperture.

12. The colostrum collection container of claim 8, wherein a visual indicator of the at least one visual indicator is oriented at an angle relative to a vertical axis of the body such that when the body is canted the visual indicator of the at least one visual indicator designates a maximum fluid capacity of the body.

13. A colostrum collection container comprising:
a body having a first opening configured to receive a breastshield and a second opening;
a receptacle sealingly disposed within the body, the receptacle having an aperture, the aperture configured to fluidly couple the first opening and the second opening of the body; and
a fluid flow path extending from the first opening through the aperture and to the second opening,
where a first fluid flows through the first opening of the body and is collected in the receptacle with gravity assistance, and a second fluid flows through the first opening, the aperture, and the second opening of the body.

14. The colostrum collection container of claim 13, wherein the body further includes a lip that extends radially outward from the first opening.

15. The colostrum collection container of claim 14, wherein the lip is integrally formed with the body.

16. The colostrum collection container of claim 13, wherein the body includes a first end and a second end opposite the first end, the first opening disposed at the first end of the body and the second opening disposed at the second end of the body.

17. The colostrum collection container of claim 13, wherein the first fluid comprises one or more of colostrum and/or milk, and the second fluid comprises at least one of air and/or milk.

18. The colostrum collection container of claim 13, wherein the receptacle is sealingly disposed in the body proximate the first opening of the body.

19. The colostrum collection container of claim 13, wherein the receptacle includes at least one visual indicator that designates a maximum fluid capacity of the body.

20. The colostrum collection container of claim 19, wherein the aperture is disposed on the receptacle such that when the first fluid in the receptacle is below the at least one visual indicator, the second fluid flows from the first opening through the aperture and to the second opening of the body.

21. The colostrum collection container of claim 19, wherein the aperture is disposed on the receptacle such that, when the first fluid in the receptacle is at or above the at least one visual indicator, the first and second fluids flow from the first opening through the aperture and to the second opening of the body.

22. The colostrum collection container of claim 13, further comprising a cap that sealingly couples to the body, the cap having a base and a sealing member that extends from the base to at least the aperture when the cap is coupled to the body thereby sealing the aperture.

23. The colostrum collection container of claim 19, wherein a visual indicator of the at least one visual indicator is oriented at an angle relative to a vertical axis of the body such that when the body is canted the visual indicator of the at least one visual indicator designates a maximum fluid capacity of the body.

24. A breastmilk expression system, the system comprising:
a breastshield;
a colostrum collection container releasably coupled to the breastshield, the colostrum collection container comprising:
a body having a first opening that receives the breastshield and a second opening disposed opposite the first opening;
a receptacle sealingly disposed within the body, the receptacle having an aperture, the aperture fluidly coupling the first opening and the second opening of the body; and
a fluid flow path extending from the first opening through the aperture and to the second opening; and
a connector coupled to the second opening of the colostrum collection container,
where, upon generation of a vacuum in the fluid flow path, a first fluid is collected in the receptacle with gravity assistance and a second fluid flows through the aperture and to the second opening.

25. The breastmilk expression system of claim 24, further comprising an overflow container that is releasably received by the connector, and a vacuum generator that is fluidly coupled to the connector and the colostrum collection container.

26. The breastmilk expression system of claim 24, wherein the colostrum collection container further includes an assembly guide that is disposed proximate the first opening of the colostrum collection container, the assembly guide extending radially outward from the first opening and configured to aid coupling of the breastshield to the colostrum collection container.

27. The breastmilk expression system of claim 26, wherein the assembly guide is integrally formed with the body of the colostrum collection container.

28. The breastmilk expression system of claim 24, wherein the body of the colostrum collection container includes a first end and a second end opposite the first end, the first opening disposed at the first end and the second opening disposed at the second end.

29. The breastmilk expression system of claim 24, wherein the first fluid comprises one or more of colostrum and/or milk and the second fluid comprises at least one of air and/or milk.

30. The breastmilk expression system of claim 24, wherein the receptacle is sealingly coupled to the body proximate the first opening.

31. The breastmilk expression system of claim 24, wherein the receptacle includes at least one visual indicator that displays a maximum fill level of the receptacle.

32. The breastmilk expression system of claim 31, wherein the aperture is disposed on the receptacle such that, when the first fluid in the receptacle is below the at least one visual indicator, the second fluid flows from the first opening through the aperture and to the second opening of the body.

33. The breastmilk expression system of claim 31, wherein the aperture is disposed on the receptacle such that, when the first fluid in the receptacle is at or above the at least one visual indicator, the first and second fluids flow from the first opening through the aperture and to the second opening of the body.

34. The breastmilk expression system of claim 24, further comprising a cap that sealingly couples to the body, the cap having a base and a sealing member that extends from the base to at least the aperture when the cap is coupled to the body thereby sealing the aperture.

35. The breastmilk expression system of claim 31, wherein a visual indicator of the at least one visual indicator is oriented at an angle relative to a vertical axis of the body such that when the body is canted the visual indicator of the at least one visual indicator designates a maximum fluid capacity of the body.

36. A method of collecting colostrum during breastmilk expression, the method comprising:
providing a colostrum collection container including a first opening, a second opening, a receptacle disposed between the first and second openings and having an aperture, and a fluid flow path including the first and second openings and the aperture; and
collecting a first fluid in the receptacle of the colostrum collection container with gravity assistance and a vacuum while a second fluid continues to flow from the first opening through the aperture and to the second opening of the colostrum collection container.

37. The method of claim 36, wherein providing the colostrum collection container further includes disposing at least one visual indicator on the body such that a visual indicator of the at least one visual indicator designates a maximum fluid capacity of the body.

38. The method of claim 37, wherein collecting the first fluid further comprises collecting the first fluid in the receptacle when the first fluid in the receptacle is below the at least one visual indicator, while the second fluid flows through the fluid flow path from the receptacle to the second opening.

39. The method of claim 37, wherein collecting the first fluid further comprises collecting the first fluid in the receptacle until the first fluid in the receptacle is at or above the at least one visual indicator, at which time at least one of the first fluid and the second fluid flows through the fluid flow path from the receptacle to the second opening.

40. The method of claim 36, further comprising coupling a cap to the body of the colostrum collection container, the cap having a base and a sealing member that extends from the base to at least the aperture when the cap is coupled to the body thereby sealing the aperture.

41. The method of claim 36, wherein providing the colostrum collection container further includes disposing a visual indicator on the body that is oriented at an angle relative to a vertical axis of the body such that when the body is canted the visual indicator designates a maximum fluid capacity of the body.

42. The method of claim 36, further comprising creating a vacuum in a portion of the fluid flow path and selectively permitting fluid flow from the first opening to the second opening of the colostrum collection container via the aperture and the vacuum.

43. A method of assembling a colostrum collection container, the method comprising:
providing a body with a first opening and a second opening;
disposing a receptacle within the body and coupling a portion of the receptacle to the body, the receptacle including a wall with an aperture; and
forming a fluid flow path including the first and second openings and the aperture of the receptacle,
such that when a vacuum is generated in the fluid flow path, a first fluid is permitted to be collected in the receptacle and a second fluid is permitted to flow through the aperture and to the second opening.

44. The method of claim 43, further comprising disposing an assembly guide proximate to the first opening of the body.

45. The method of claim 44, further comprising coupling a breastshield to the first opening of the body using the assembly guide.

46. The method of claim 43, further comprising disposing at least one visual indicator on the body such that a visual indicator of the at least one visual indicator designates a maximum fluid capacity of the body.

47. The method of claim 46, wherein forming the fluid flow path comprises forming the aperture on the receptacle such that when the first fluid in the receptacle is below the at least one visual indicator, the second fluid flows from the first opening through the aperture and to the second opening of the body.

48. The method of claim 46, wherein forming the fluid flow path comprises forming the aperture on the receptacle such that when the first fluid in the receptacle is at or above the at least one visual indicator, the first fluid and the second fluid flow from the first opening through the aperture and to the second opening of the body.

49. The method of claim 43, further comprising coupling a cap to the body, the cap having a base and a sealing member that extends from the base to at least the aperture when the cap is coupled to the body.
